(19) United States
(12) Patent Application Publication
(10) Pub. No.: US 2021/0386813 A1
(43) Pub. Date: Dec. 16, 2021

(54) COMPOSITION FOR TREATING OVERWEIGHT AND/OR OBESITY

(71) Applicant: NEXIRA, ROUEN (FR)

(72) Inventor: Damien GUILLEMET, ROUEN (FR)

(73) Assignee: NEXIRA, ROUEN (FR)

(21) Appl. No.: 17/287,200

(22) PCT Filed: Oct. 22, 2019
(86) PCT No.: PCT/EP2019/078746

§ 371 Date: Apr. 21, 2021

(30) Foreign Application Priority Data
Oct. 22, 2018 (FR) 1859711

(51) Int. Cl. A61K 36/738; A61K 9/00; A61K 36/48; A61K 36/23; A61K 36/28; A23L 33/105; A23K 10/30; A23P 10/40; A23K 40/10

(52) U.S. Cl. CPC A61K 36/738; A61K 9/0053; A61K 36/48; A61K 36/23; A23V 2002/00; A23L 33/105; A23K 10/30; A23P 10/40; A23K 40/10; A61K 36/28

(57) ABSTRACT

The invention relates to a composition comprising an edible extract of at least two of the plants selected from the group consisting of Acacia catechu, Daucus carota, Rosa canina, and Helichrysum italicum. The composition and the products derived therefrom are modulators of adipose tissue or fat burners, in particular by targeted activation of thermogenesis in a human or animal subject.

### INVENTIVE FIELD

The present invention relates to a food supplement composition comprising at least one edible plant extract. The invention also relates to the use of this composition for treating overweight and/or obesity.

### BACKGROUND

The management of fats in the human body is regulated by the adipose tissue, and more particularly by the adipocytes that are the cells that make up this tissue. This regulation involves a plurality of cellular mechanisms, among which are mechanisms for the storage of fats, and mechanisms for the release, breakdown and/or transformation of fats.

In particular, lipogenesis, lipolysis, adipogenesis and thermogenesis exist.

Lipogenesis essentially comprises the transformation of free fatty acids into triglycerides for storage in the adipocytes. Lipolysis essentially comprises the hydrolysis of stored triglycerides to release and evacuate (that is to say re-release into the blood stream) fats from the adipocytes. Adipogenesis is the cellular mechanism wherein cells known as preadipocytes differentiate into adipocytes in order to increase the storage of fats in the body.

In thermogenesis, the adipocytes transform fats directly into heat. More generally, thermogenesis is the production of heat of the human body by increasing the cellular metabolism. Thermogenesis occurs particularly when the temperature of the body is below the set point, which is approximately 37° C. Thermogenesis takes place, at least partly, in a certain class of adipocytes known as brown adipocytes. In practice, this concerns a transformation of fats into heat, cf. Fenzl A., Kiefer F W., Brown adipose tissue and thermogenesis, Horm Mol Biol Clin Investig., 2014, 19(I):25-37.

Generally, the metabolism of adipocytes has a major effect on the overall metabolism of the human body. People having an excess of adipose tissue thus generally have health problems. More globally, the excess of fatty mass results in drawbacks for health. This excess is known as overweight and may lead to obesity, cf. Brinderjit Kaila and Maitreyi Raman, MD FRCPC Obesity: A review of pathogenesis and management strategies, Can J Gastroenterol, 2008, 22(1):61-68 and Lean M. et al., Making progress on the global crisis of obesity and weight management, BMJ, 2018, 361. Currently, approximately 30% of the population is overweight and obese.

The prior art describes plants having an effect on the adipose tissue.

In particular, coffee and tea have effects on the adipose tissue. The effects are mainly related to the presence of caffeine in these plants. Caffeine is particularly involved in the activation of lipolysis and the general stimulation of the metabolism of adipocytes, cf. M.-H. Pan, Y.-C. Tung, G. Yang, S. Li, C.-T. Ho, Molecular mechanisms of the anti-obesity effect of bioactive compounds in tea and coffee, 2016, Food Funct., 2016, Nov. 9; 7(11):4481-4491.

Furthermore, green coffee has the advantage of including polyphenols, which seems to promote at least partly thermogenesis. Consequently, green coffee is applicable in the treatment of overweight or obese people.

However, the daily dosage relating to caffeine is difficult to control. This is particularly due to the presence of this molecule in many foods. A precise monitoring is difficult, or even impossible. In addition, beyond its effect on the adipose tissue, caffeine has effects on other metabolic processes in the human. Generally, caffeine is known to be a stimulant and a psychostimulant. It has an effect on the cardiovascular system that may result in a harmful acceleration of the heart rate. In some cases, caffeine has a diuretic effect. Furthermore, it is antagonist of the receptors of the adenosine neurotransmitter in the central nervous system. Thus, caffeine may generate physical and psychological dependencies in certain people.

All of this means that coffee or tea are not entirely satisfactory for the treatment of overweight or obese people. In addition, caffeine stimulates lipolysis, that is to say the process of hydrolysis of triglycerides stored in the adipocytes to release and evacuate fats. In the body, the fats are therefore released by the adipocytes into the general blood stream. This may particularly result in cardiovascular problems or hepatic dysfunctions.

Other plants seem to have an effect on the metabolism of the adipose tissue.

Document EP 3 096 767 discloses a composition comprising okra. The composition reduces the absorption of fats by the body. For this, the composition must be taken during meals, or at least be taken close to meals (15 to 30 minutes before, or 30 to 45 minutes after the meal).

There is a constant need to identify new compounds having an effect on the adipose tissue.

Very particularly, there is a need to identify compounds capable of activating or stimulating thermogenesis. Indeed, thermogenesis transforms the fats into heat Therefore, it does not have any particular drawbacks related to lipolysis.

The present invention improves the situation.

### SUMMARY

To this end, the aim of the invention is a composition comprising an edible extract of at least one of the plants selected from the group consisting of Nelumbo nucifera, Mangifera indica, Acacia catechu, Commiphora mukul, Daucus carota, Cinnamomum verum, Rosa canina, and Helichrysum italicum.

In one embodiment, the composition may comprise an edible extract of at least two of said plants.

In other embodiments, the composition comprises an edible extract of at least three of said plants or an edible extract of four of said plants.

In one embodiment, the composition comprises an edible extract of two of said plants thus forming a combination of plant extracts, said combination being selected from:
a combination of Acacia catechu and Commiphora mukul;
a combination of Acacia catechu and Helichrysum italicum;
a combination of Nelumbo nucifera and Helichrysum italicum;
a combination of Daucus carota and Rosa canina;
a combination of Acacia catechu and Rosa canina; and
a combination of Daucus carota and Cinnamomum verum.

In one embodiment, the edible extract or extracts respectively come:
from the flower of Nelumbo nucifera;
from the skin of the fruit of Mangifera indica;
from the bark of Acacia catechu;
from the exudate of Commiphora mukul;
from the seed of Daucus carota;
from the leaf of Cinnamomum verum;
from the fruit of Rosa canina;
from the aerial part of Helichrysum italicum.

The composition may be for use in the treatment and/or the prevention of an affection selected from overweight, obesity and a metabolic disease in a human or an animal.

The edible extract or extracts may be in powder or granular form whereof the particle size diameter d90 of the particles is less than or equal to 180 μm.

The composition may comprise 0.001% to 99% by weight, preferably 10% to 80% by weight, of the edible extract or extracts.

The composition may be in a form selected from a tablet, a cachet, a capsule, a granule, a gelatin capsule, a sugar-coated tablet, a chewing gum, a paste, a beverage, a syrup and a powder.

The invention also relates to a foodstuff, or food supplement, or dietary supplement, or meal replacement product, or beverage, or beverage supplement, or pharmaceutical product, comprising the above-described composition.

In one embodiment, the pharmaceutical product further comprises one or more supports and/or excipients.

Furthermore, the aim of the invention is a composition or a product such as defined above for the activation of thermogenesis in a human or animal subject.

The aim of the invention is also a non-therapeutic method for controlling the body weight of a subject, said method comprising the administration of an effective quantity of a composition such as defined above.

### BRIEF DESCRIPTION OF THE DRAWINGS

Other advantages and features of the invention will become apparent upon reading the following detailed description and in the appended drawings wherein:

FIG. 1 shows a diagram of a permeability test system;

FIG. 2 shows a table comprising plant extracts and their respective effects on a part of the adipose metabolism; and

FIG. 3 shows a table comprising combinations of plant extracts and their respective effects on a part of the adipose metabolism.

### DETAILED DESCRIPTION OF THE PREFERRED EMBODIMENTS

The figures, tables and the description hereafter mainly contain elements of certain character. The figures and tables are an integral part of the description, and may not only be used to better understand the present invention, but also to contribute to its definition, if applicable.

In the present description, “active ingredient” means a molecule or a combination of molecules having a beneficial effect on the treatment for overweight or obesity. In particular, this concerns a molecule or a combination of molecules having an effect on the adipocytes so as to engage or cause:
thermogenesis,
lipolysis,
the inhibition of adipogenesis, and/or
a lipid anti-accumulation effect.

In the present description, “overweight” subjects or people means subjects or people whose body mass index (BMI) is between 25 and 30.

In the present description, “obese” subjects or people means subjects or people whose body mass index (BMI) is greater than or equal to 30.

Overweight and obesity are sometimes qualified as “disease” in order to designate their nature corresponding to a health disorder.

In the present description, the term “edible part” of plant designates a part of plants able to be ingested by a human per os, that is to say by oral route or more generally by gastro-intestinal route. Such an “edible part” may further be ingested by an animal. The composition of the invention comprises extracts of edible parts of plants.

Generally, a plant has two main parts: an underground part that in particular comprises the root (or the tubercle or the rhizome), and an aerial part that in particular consists of the stem, leaves, buds, fruits and/or flowers.

An edible part of a plant may be in solid or liquid form. Therefore, it may particularly concern leaves, a root, a stem, a fruit, flowers or also the exudate or sap of a plant, transformed or not. It may concern all or only one or more parts of these elements.

The extract may be raw or be from an extraction process usually used in the field of food supplements.

Such extraction processes may particularly comprise a dehydration or a cold drying so that the edible part is transformed into a format suitable for human consumption. The process may further comprise the crushing or the cutting of certain parts of the plants. Plant extracts may thus be found in particular in powder or granule form. The process may further comprise the washing, the disinfection, the discolouration, the drying and/or the cooking of plant parts. Plant extracts may therefore also be for example in suspension, aqueous solution or dispersion form. Extracts may further be in “plant totum” form. A plant totum corresponds to an entire plant, or a plant part, dehydrated by drying and crushing.

“Edible extract” designates the edible part or parts of the plant, transformed or not by an extraction process, present in the composition of the invention.

In embodiments, the composition of the invention comprises solid particles of plant extracts. The average size of the particle diameter is generally less than 500 μm, preferably less than 400 μm, more preferably less than 300 μm, still more preferably less than 150 μm. In a preferable embodiment, the average size of the particle diameter is between approximately 180 μm and approximately 90 μm. The particle size is expressed in distribution d90, that is to say that 90% of the particles have a diameter less than the value determined by the granulometer.

In embodiments of the invention, the composition may comprise dietary fibres. For example the composition may comprise polysaccharides such as polysaccharides other than starch. These types of polysaccharides are not generally broken down in the gastro-intestinal tract. This is at least partially due to the incapacity of the human body to hydrolyse these polysaccharides. One advantage of including dietary fibres in the composition of the invention resides in the fact that these fibres are generally capable of binding to edible fats present in the gastro-intestinal tract and of evacuating them from the human body in form of complex fibres/fats via the excrements. This may partly contravene the assimilation and the absorption of edible fats by the body.

In other embodiments of the invention, the composition may comprise more or less soluble dietary fibres. By way of example, it may be cited chitosan, acacia gum, guar gum, pectin, oat flakes, wheat, soybean fibres, beetroot extracts, cellulose, or any derivative of the latter.

In yet other embodiments of the invention, the composition of the invention may be coupled with satiety agents, draining products, digestion enzyme inhibitor products, prometabolic products, probiotic products, or prebiotic products. The composition of the invention may be qualified as phytoactive product. It may further be coupled with other phytoactive products such as polyphenols or terpenes. A main function of the invention is to act as a product for modulating adipose tissue.

The composition of the invention may be in different forms suitable for ingestion by oral route. In particular the composition may be in the form of tablets, gelatin capsules, capsules, powder, liquid or also in the form of syrup.

Thus, the composition of the invention may comprise carrier substances pharmaceutically acceptable and/or compatible with the agrifood market. The composition may further comprise ingredients of the type diluent, adjuvant, excipient, preservative, filler, disintegrating agent, wetting agent, emulsifier, suspending agent, taste intensifying agent, aromatic agent, odorant agent, antibacterial agent, anti-yeast agent, lubricant and dispersing agent. Formulation techniques are particularly described in the work Remington: The Science and Practice of Pharmacy, 19th Edition, ISBN-13: 978-0912734040 or in the work Conception des complements alimentaires: Marché, développement, réglementation et efficacité, ISBN-13: 978-2743022211.

When the composition of the invention is in solid or semi-solid form (including in gum form) the carrier substance may be dicalcium phosphate. The diluents and fillers may include starch, lactose, sucrose, glucose, fructose, mannitol, maltodextrin or microcrystalline cellulose. The binding agents may include agents of the type hydroxylepropyl cellulose, hydroxylepropyl methylcellulose, carboxymethyl cellulose, gelatin, polyvinylpyrrolidone, polyvinyl acetate, sucrose and acacia gum. The disintegrating agents may include agents of the type starch, sodium starch glycolate, calcium carbonate, alginic acid or silicate. The lubricants may include talc, calcium stearate, magnesium stearate, stearic acid, sodium sulphate, silica or sodium fumarate. The solubilising agents may include sodium lauryl sulphate.

When the composition is for example in the form of tablets, gelatin capsules or capsules, a controlled release over time of the active ingredient may be provided. The controlled release profile may be adapted according to the needs targeted, and this particularly by means of synthetic and/or natural polymers. Moreover, a controlled release may be obtained by means of microspheres or liposomes.

When the composition of the invention is in the form of liquid or in the form of syrup, including suspension or emulsion type solutions, it may comprise water, propylene glycol, or alcohol. It is possible to include a humectant such as glycerol, or sweetening agents such as liquid glucose, aspartame or stevia.

The composition of the invention is very particularly intended for the food supplement market. The composition of the invention is also intended for the pharmaceutical market. The composition is further intended for the food formulation market.

In particular, the invention relates to a composition of foodstuff, food supplement, dietary supplement, meal replacement product, beverage, beverage supplement, and pharmaceutical product in any usual form.

The composition of the invention may be included in convenience foods. In embodiments, these convenience foods comprise 0.001% to 50% by weight of the composition of the invention. In particular embodiments, the convenience foods comprise 0.001% to 40% by weight, 0.001% to 30% by weight, 0.001% to 20% by weight, 0.001% to 10% by weight, 000.1% to 5% by weight or 000.1% to 2% by weight of the composition of the invention. Advantageously, the convenience foods comprise at least 0.1% to 5% by weight or more than 5% by weight of the composition of the invention. By way of example, convenience foods may be cited such as ready meals, cereal bars, confectionary products, beverages including shot drinks, snacks or biscuits.

The composition of the invention may be administered per os at any time in order to develop its effects. It may also be taken at the time of daily meals in view of controlling body weight. In embodiments, the ingestion of the composition is carried out 15 to 30 minutes before or after the meal.

The dosage of the composition of the invention depends substantially on the subject that ingests it. Thus for therapeutic applications, the dosage of the composition varies in particular the weight and the body mass index (BMI) of the subject. The determination of the dosage may be determined by the person skilled in the art.

In general, the dosage of the composition may reach up to 10 g per day. In embodiments, the formulation of the composition of the invention is adapted to allow a daily dosage of 100 mg at approximately 7.5 g. In other embodiments, the formulation is adapted to allow a daily dosage of 200 mg at 5 g or of 300 mg at 3 g. Typically, a daily dosage of 100 mg is provided.

The composition of the invention comprises a plant extract or a combination of plant extracts. According to one embodiment of the invention, the composition comprises at least 1% by weight of the plant extract or of the combination of plant extracts. In another embodiment, the composition includes 1 to 99% by weight of the plant extract or of the combination of plant extracts. In other embodiments, the composition comprises at least 5% by weight, 10% by weight, 20% by weight, 30% by weight, 40% by weight, 50% by weight, 60% by weight, 70% by weight, 80% by weight or at least 90% by weight of the plant extract or of the combination of plant extracts.

The composition of the invention may further comprise various types of nutrients such as vitamins and/or minerals. The vitamins may particularly be selected from vitamin A, vitamin D, vitamin E, vitamin K, thiamine, riboflavin, vitamin B12, the carotenoids such as beta-carotene is zeaxanthin, folic acid, vitamin B8, vitamin C, or a combination thereof. The minerals may particularly be selected from calcium, magnesium, iron, zinc, manganese, copper, iodine, potassium, molybdenum, sodium, selenium, fluorine or chlorine. According to embodiments, the nutrients may be present in the composition ranging up to a content of approximately 90% or 50% by weight.

The conditioning or the formulation of the food supplement composition according to the invention may be performed according to the methods known in the agrifood industry. These methods may particularly comprise mixing, cooking, extrusion, fermentation, molding, pressing, drying and shaping steps.

In embodiments, a method for preparing the composition comprises the following steps:
washing an edible part of plant;
cutting the edible part;
drying the edible part;
crushing the edible part so as to obtain a powder.

A preparation method may also comprise operations of the type solid/liquid extraction, liquid/liquid purification, absorption resin purification, membrane purification, filtration, concentration, pasteurization, and/or spray drying, vacuum or also lyophilization.

The Applicant developed a two-step analysis to evaluate the effect of products coming from plants (or plant derivatives) on the adipose tissue, and in particular the effect of these products on thermogenesis.

The 1^{st} step mainly includes a bioavailability study of products, or their combinations, on cells named Caco-2. In this step, the fraction of the product (or the molecular part) that is assimilated by the human organism after an administration per os is isolated. For example, when a product includes an active ingredient this may concern the intact fraction of this active ingredient that reaches the general blood stream. In other examples, this fraction may involve associating molecules from the product, or may consist of molecules that have undergone a biochemical transformation or cellular metabolization.

The 2^{nd} step includes in particular a study of the effect of the bioavailable fraction on human preadipocytes or human adipocytes.

The use of the bioavailable fraction on preadipocytes and adipocytes makes the evaluation significative on the biological level. In particular, it drastically increases the in vivo representativeness.

In order to perform the 1^{st} step the Applicant took advantage of a test based on the use of Caco-2 cells.

The Caco-2 cells constitute a human tumoral cell line (immortalized cells) of intestinal origin isolated from a colon adenocarcinoma.

In certain culture conditions, these cells have the capacity to differentiate spontaneously into intestinal cells organized into adjacent and polarized monolayers in order to form an epithelium imitating a functional intestinal barrier. The epithelium may particularly have microvilli, tight junctions, carriers specific to the intestinal system, and enzymes of the metabolization process, cf. Pinto M. et al., Enterocyte-like differentiation and polarization of the human colon carcinoma cell line CACO-2 in culture, Biol Cell, 1983, 47:323-30, and Hidalgo I J. et al., Characterization of the human colon carcinoma cell line (Caco-2) as a model system for intestinal epithelial permeability, Gastroenterology, 1989, 96(3):736-49.

Tests on Caco-2 cells thus make it possible to evaluate the intestinal permeability of a product. In other words, tests on Caco-2 provide information on the capacity of a compound to pass through the intestinal wall and, if applicable on its biochemical transformation, before re-joining the blood stream and lymphatic circulation and being distributed into the organism.

Here a Caco-2 test, called permeability test is used to obtain the bioavailable fractions of plant extracts of the invention. This concerns an implementation on isolated compartment monolayer insert.

For this, Caco-2 cells are cultivated on a microporous membrane placed in individual culture chambers. After a culture period of approximately 20 to 25 days, the cells form a differentiated monolayer isolating the upper and lower compartments of the culture chamber.

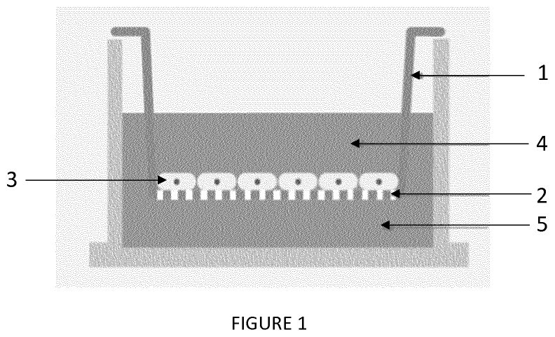

FIG. 1 shows a diagram of an intestinal permeability test system including a Caco-2 cell culture. This concerns a test for studying bioavailability through intestinal epithelium.

The system includes a Transwell® insert 1 available from Merck. The insert comprises a microporous membrane 2 on which Caco-2 cells 3 are cultivated in monolayer. The microporous membrane/Caco-2 cell assembly separates an apical compartment 4 and a basolateral compartment 5.

Here, the microporous membrane is arranged in six wells. Each well has a diameter of 24 mm and a microporous membrane base having pores of 0.4 μm. Each well is inoculated with a cell density of 25×10⁴ cells/well. The culture medium is changed every day for 21 days in order to obtain the cellular monolayer, cf. Reboul et al., Lutein transport by Caco-2 TC-7 cells occurs partly by a facilitated process involving the scavenger receptor class B type I (SR-BI), Biochem J, 2005, 387(Pt 2):455-61.

The apical and basolateral compartments are filled respectively with 1.5 ml and 2.5 ml of complete medium. An exposure of the cells to the plant extracts is performed for a period of approximately 24 hours. The cells and the respective culture media of the apical and basolateral compartments are harvested and analyzed. Optionally, the respective cells and culture media of the apical (apical medium) and basolateral (basolateral medium) compartments are frozen at −80° C. for subsequent experiments.

The use of such a system makes it possible to have access to the apical side (side higher than the cells, imitating the intestinal lumen) and to the basal side (side lower than the cells, imitating the internal environment) of the intestinal epithelium. This makes it possible to evaluate product exchanges between the two sides (apical/basal). Thus generally, the permeability test on Caco-2 cells makes it possible to investigate particularly:
the permeability of the intestinal epithelium in relation to a product of interest;
the bioavailability expected in the human;
the screening and the selection of products of interest;
the absorption kinetics of the intestinal system; and
the biological study of absorption mechanisms.

After exposure of the Caco-2 cells to plant extracts or combinations of plant extracts introduced into the apical medium, it is then possible to harvest the basolateral medium (BM) comprising the bioavailable fraction, from the extracts or their combinations. It will be noted that the BM may further contain metabolites resulting from the specific exposure of the Caco-2 cells to the plant extracts. The BM is generally harvested and stabilized by −80° C. freezing.

The Applicant thus evaluated the bioavailability of a multitude of plant extracts in order to subsequently evaluate the effect of the bioavailable fractions on mature preadipocytes or adipocytes.

The evaluation of the effect of each bioavailable fraction includes particularly:

(I) a cytotoxicity study;
(II) a preadipocyte differentiation and lipid accumulation (adipogenesis) study;
(III) an adipose tissue phenotypic orientation (thermogenesis) study;
(VI) an adipose tissue energy metabolism study; and
(V) a lipolysis study.

Each evaluation is accompanied by one or more control samples in order to analyze the effects.

### (I) Cytotoxicity Study

We speak of cytotoxicity (or of a cytotoxic effect) when a substance has a toxic effect on cells. More particularly, cytotoxicity is the property of a chemical or biological agent to be toxic for the cells, optionally up to destroying them.

Firstly (1.1), the Applicant performed a preliminary study on the cytotoxicity of plant extracts on Caco-2 cells. Secondly (1.2), the Applicant evaluated the cytotoxicity of bioavailable plant extracts and their combinations on preadipocytes.

(I.1) Cytotoxicity Study of Plant Extracts on Caco-2 Cells

The Applicant evaluated the cytotoxic effects of a multitude of plant extracts on Caco-2 cells.

The evaluation includes an analysis of membrane damage, a morphological analysis of cells and a mortality estimation by coloring dead cells in trypan blue.

The analysis of the damage of cell membranes is performed with the CytoTox 96® Non-Radioactive Cytotoxicity Assay kit available from Promega under the reference G1780.

This test is based on the principle that cells having a damaged cell membrane release lactase dehydrogenase (LDH) into the culture medium. LDH is measured by supplying in the medium lactate, oxidized nicotinamide adenine dinucleotide (NAD+) and iodonitrotetrazolium purple as substrates in presence of diaphorase. Red formazan is then created. The red formazan is proportional to the LDH released by the cells.

Here, Caco-2 cells are inoculated at a density of 25×10³ cells/well in microtiter plates with 96 wells. The medium is changed every day for 21 days in order to obtain a monolayer of cells, cf. Reboul et al., Lutein transport by Caco-2 TC-7 cells occurs partly by a facilitated process involving the scavenger receptor class B type 1 (SR-81), Biochem J, 2005, 387(Pt 2):455-61. The cells are subsequently incubated with concentrations of extracts (variable according to the extracts selected) in 2% of fetal bovine serum medium for a period of 48 hours. The medium is changed after 24 hours. After incubation, the cell culture medium is harvested and the LDH level is quantified.

(I.2) Cytotoxicity Study of Bioavailable Plant Extracts on Human Preadipocytes

The Applicant evaluated the cytotoxic effects of a multitude of bioavailable plant extracts, as well as the cytotoxic effects of the combination of these extracts, on preadipocytes coming from various donors.

The extracts are harvested or derived from the edible parts of the respective plants.

The cytotoxic effects of bioavailable plant extracts and their combinations was evaluated on preadipocytes. Two technical approaches were implemented:
the evaluation of the cellular viability by the measurement of the production of formazan by active (therefore living) cells; and
the analysis of the damage of cell membranes by the measurement of lactate dehydrogenase (LDH) in the cell culture medium.

The evaluation of the cellular viability is performed with the Cell Titer 96® AQueous One Solution Cell Proliferation Assay test kit available from Promega under the reference G3580.

In this test, the compound MTS-tetrazolium (3-(4,5-dimethylthiazol-2-yl)-5-(3-carboxymethoxyphenyl)-2-(4-sulfophenyl)-2H-tetrazolium) is transformed by active (living) cells in a culture medium in colored formazan. The transformation of MTS-tetrazolium is performed by the mitochondria in the cells. Formazan is soluble in the culture medium. The quantity of formazan is directly proportional to the number of living cells and may be measured by spectrophotometry (absorbance).

In practice, the cellular viability test is therefore based on the mitochondrial activity of the cells.

As a result, the viability study may provide precious information for the energy metabolism study (IV). Indeed, when the cells are viable, this test provides information on the mitochondrial activity of the cells.

The analysis of the damage of cell membranes is performed with the CytoTox 96® Non-Radioactive Cytotoxicity Assay test kit available from Promega under the reference G1780 (see above I.1).

In order to evaluate the cytotoxicity of a plant extract or of a combination of plant extracts on the cells, the extract or the combination of extracts is compared with a control.

The control may consist of cells that have not been exposed to a plant extract. The control may also consist of cells that have been exposed to a known non-cytotoxic plant extract.

An extract is considered as cytotoxic if the LDH level measured is greater than or equal to 20% in relation to the LDH level measured with the control extract.

A statistical analysis is performed. The results are expressed in percentages in relation to the control extract; except for the LDH test, for which the results are expressed as percentages of the cell lysis control (average+/− SEM), which represents the positive control for the cytotoxicity (the control is fixed at 100%). Statistical analyses are carried out by using the Prism software available from GraphPad Software.

The Applicant obtained adipose tissue donations, each time, from three different donors. For a sufficiently representative statistical analysis, a use of triple values was implemented per experiment (for a SEM—Standard Error of Mean). This results in a total of nine values for each experiment. Comparisons between the treatment with plant extract(s) and the controls (without treatment with extract) were analyzed with the Friedman test. This makes it possible to compare repeated data for dependent samples. The Friedman test is a non-parametric test that may be used when we are in presence of n paired samples corresponding to n treatments concerning the same type of cells, in order to highlight a difference between the treatments.

The Friedman test is followed by a Wilcoxon test (Wilcoxon-Mann-Whitney) in order to compare paired samples, two by two. Differences were considered as statistically significative when p 0.05 (*p 0.05, **p 0.01, ***p 0.001, ****p 0.0001), p being the probability value (p-value).

Preadipocytes coming from humans are used for the evaluation of the cytotoxicity of the plant extracts or of the combination of two or more of said extracts.

The three adipose tissue donors are adult females respectively 27, 32 and 34 years old undergoing a cosmetic or reconstructive surgery intervention. The donors had a respective body mass index (BMI) of 23.3, 26.7 and 29.3.

The preadipocytes are prepared from an enzymatic digestion of samples of the subcutaneous adipose tissue of the donors, followed by a centrifugation and sampling of the cellular pellet.

The preadipocytes are subsequently spread at low density on microtiter plate 96 and incubated for 5 to 6 days in combination with a basolateral medium (BM) of Caco-2 cells exposed to extracts or combinations of extracts, at a 1:8 dilution each one in a medium comprising 1% of fetal bovine serum (FBS). This results in a 1:4 dilution of the total basolateral medium in the culture medium.

The 1:8 dilution is performed to mitigate a biochemical activity inherent to the virgin basolateral medium, that is to say a basolateral medium whereof the Caco-2 cells have not been exposed to a plant extract. Indeed, a 1:8 dilution makes it possible to diffuse almost all of the activity specific to the virgin basolateral medium. The measurements are thus more sensitive and representative.

The culture medium is changed every 48 hours. Each old culture medium is sampled and harvested for the cytotoxicity analysis.

The cellular viability is measured directly in the wells of the microtiter plate.

Here, the various basolateral media of the preliminary study (1.1) are used. These media include each time the bioavailable fraction of the respective extracts. The mixture of various basolateral media makes it possible to test the combination of bioavailable fractions of various extracts.

The concentrations retained have no cytotoxic activity towards the cells.

### (II) Preadipocyte Differentiation and Lipid Accumulation (Adipogenesis) Study

The Applicant evaluated the effects of a multitude of bioavailable plant extracts, as well as the effects of the combination of these extracts, on the preadipocyte differentiation and on the lipid accumulation. The preadipocytes come from various donors.

The extracts are harvested or derived from the edible parts of the respective plants.

In overweight or obese patients an increase of the adipose tissue is noted. This is particularly due to the increase in size of the cells forming this tissue. More specifically, the preadipocytes present in the adipose tissue are responsible by way of adipogenesis of the increase of the number of adipocytes. Adipogenesis is the cellular differentiation process wherein preadipocytes become adipocytes. Preadipocytes are fibroblast-like cells, whereas adipocytes are generally round-shaped voluminous cells including a relatively voluminous lipid droplet, cf. Lowe C. E. et al., “Adipogenesis at a glance”, J Cell Sci, 2011, 124:2681-2686.

In practice, the effects of the bioavailable extracts on the lipid accumulation are studied by measuring the accumulation of lipid droplets in the differentiated preadipocytes, that is to say in the adipocytes. The measurement is optical and is performed by fluorescence.

The statistical analysis is performed under the same conditions as for the cytotoxicity study (I).

Preadipocytes coming from humans are used for the lipid accumulation study.

The three adipose tissue donors are adult females respectively 26, 41 and 45 years old undergoing a cosmetic or reconstructive surgery intervention. The donors had a respective body mass index (BMI) of 22.2, 22.9 and 26.8.

The preadipocytes are isolated by digestion and centrifugation in accordance with the protocol described for the cytotoxicity study (I) above.

The preadipocytes are spread at high density on microtiter plate 96 and incubated with a basolateral medium of Caco-2 cells exposed to extracts or combinations of extracts for a period of approximately 10 to 12 days, cf. Hauner H., Skurk T. and Wabitsch M., “Cultures of Human Adipose Precursor Cells”, Method in Molecular Biology, 2001, 155:239-247. Each lot of basolateral medium exposed to one or more extracts is diluted at a rate of 1:8 in a differentiation medium before placing in contact with the preadipocytes. This dilution is carried out to mitigate the activity of the virgin basolateral medium (see above). The culture medium is changed every 48 hours (placed back in contact with the BM). After the culture, the differentiated cells are fixed with a 4% paraformaldehyde solution then colored with boron dipyrromethene (BODIPY® available from ThermoFisher Scientific under the reference D3922) and 4′,6-diamidino-2-phenylindole (DAPI Invitrogen Molecular Probes® available from ThermoFisher Scientific under the reference D1306). The coloring makes it possible in particular to observe the lipid droplets and the cell nucleus.

The quantification is based on fluorescence intensity measurements on images. The acquisition and the processing of images includes particularly:
the acquisition of microphotographs with a video microscope using the so-called Apotome technology (7 micrographs per well, 3 wells per processing condition);
the quantification of the lipid accumulation by the treatment and the image analysis of the ImageJ software developed by National Institutes of Health (NIH);
the determination of the number of nuclei by means of a shape detection and shape sorting algorithm based on the nucleus size and the circularity of the nucleus;
the connection between the fluorescence detected and the number of nuclei.

### (III) Adipose Tissue Phenotypic Orientation (Thermogenesis) Study

The Applicant evaluated the effects of a multitude of bioavailable plant extracts, as well as the effects of the combination of these extracts, on the differentiation of preadipocytes or adipocytes into beige adipocytes. More generally the Applicant evaluated the orientation of a white phenotype towards a beige phenotype.

Adipose tissue includes two different types: white adipose tissue and brown adipose tissue.

White adipose tissue mostly comprises white adipocytes and is involved in the storage and the mobilization of energy. Brown adipose tissue mostly includes brown adipocytes and is involved in thermogenesis.

Brown adipose tissue is not generally predominant in adult humans. However, white adipose tissue may include brown adipocytes after a thermogenesis stimulation of this tissue, for example after an exposure to cold. These brown adipocytes included in the white adipose tissue are qualified as beige adipocytes (or brite adipocytes coming from brown in white).

The brown and beige adipocytes have common features. In particular, these adipocytes include a large number of mitochondria. Mitochondria play a key role in the energy metabolism of the adipose tissue. Furthermore, brown and beige adipocytes express uncoupling protein 1 (UCP1). Uncoupling protein 1 is present in mitochondria and plays an essential role in thermogenesis, cf. Estève D., Boulet N. et al. “Human white and brite adipogenesis is supported by MSCA1 and is impaired by immune cells”, Stem Cell, 2015, 33(4):1277-91.

The statistical analysis is performed under the same conditions as for the cytotoxicity study (I).

Preadipocytes coming from humans are used for the cellular differentiation study. The three donors are those of the cytotoxicity study (I) above.

The preadipocytes are isolated by digestion and centrifugation in accordance with the protocol described for the cytotoxicity study (I) above. The preadipocytes are spread at high density on microtiter plate 96 and cultivated according to the protocol described for the lipid accumulation study (II). The placing in contact with the basolateral medium is performed according to the protocol described for the lipid accumulation study (II).

After the culture, the differentiated cells are fixed with a 4% paraformaldehyde solution then colored with an anti-UCP1 fluorescent antibody available from Sigma-Aldrich under the reference U6382. Furthermore, the cells are colored with boron dipyrromethene (BODIPY® available from ThermoFisher Scientific under the reference D3922) and 4′,6-diamidino-2-phenylindole (DAPI Invitrogen Molecular Probes® available from ThermoFisher Scientific under the reference D1306). The coloring makes it possible in particular to observe UCP1, the lipid droplets and the cell nucleus.

The quantification is based on fluorescence intensity measurements on images. The acquisition and the processing of images includes particularly:
the acquisition of microphotographs with a video microscope using the so-called Apotome technology (7 micrographs per well, 3 wells per processing condition);
the UCP1 quantification and lipid accumulation by the treatment and the image analysis of the ImageJ software;
the determination of the number of nuclei by means of a shape detection and shape sorting algorithm based on the nucleus size and the circularity of the nucleus;
the acquisition of data on the UCP1 expression in the fluorescent zones in relation to the total number of nuclei and to the number of nuclei of differentiated cells (UCP1 mostly able to be expressed in the latter).

(IV) Adipose Tissue Energy Metabolism Study

The Applicant evaluated the effects of a multitude of bioavailable plant extracts, as well as the effects of the combination of these extracts, on the energy metabolism of preadipocytes coming from various donors.

In humans, oxidative phosphorylation takes place at the membrane of the mitochondria. In sum, this concerns the phosphorylation of ADP into ATP thanks to the energy released by the oxidation of electron donors by the respiratory chain.

In order to study the energy metabolism, the ATP production is measured in the differentiated preadipocytes. This provides information on the presence of mitochondria and their activity. Here, the Mitochondrial ToxGlo® test available from Promega under the reference G8000 is used.

The test includes placing preadipocytes in contact with an ATP detection reagent. This results in the cell lysis of the preadipocytes and the generation of a light signal proportional to the ATP quantity. The ATP detection reagent comprises luciferin, ATPase inhibitors and Ultra-Glo® thermostable luciferase. The values are normalized with a DAPI quantification. The Normalization is carried out exclusively in relation to the basolateral medium.

The statistical analysis is performed under the same conditions as for the cytotoxicity study (I).

Preadipocytes coming from humans are used for the energy metabolism study. The three donors are those of the cytotoxicity study (I) above.

The preadipocytes are isolated by digestion and centrifugation in accordance with the protocol described for the cytotoxicity study (I) above. The preadipocytes are spread at high density on microtiter plate 96 and cultivated according to the protocol described for the lipid accumulation study (II). The placing in contact with the basolateral medium is performed according to the protocol described for the lipid accumulation study (II).

The quantification of the ATP synthesis is performed on differentiated cells.

(V) Lipolysis Study of Mature Adipocytes Exposed to Bioavailable Plant Extracts

White adipose tissue is a major source of energy for the human body. The adipocytes store triglycerides (also known as triacylglycerols, triacylglycerides or TAG). Under bodily effort, the triglycerides are transformed into non-esterified fatty acids (NEFA) and into glycerol. This makes it possible to re-release the transformed triglycerides into the cells and into the blood stream.

This transformation process is known as lipolysis. Lipolysis is particularly triggered by catecholamines (in particular by adrenaline and noradrenaline). Lipolysis is further regulated by hormone-sensitive lipase (HSL).

The quantification of the production of glycerol is performed by means of the Glycerol Assay Kit test kit available from Randox under the reference GY105.

The quantification of the production of non-esterified fatty acids is performed by the NEFA HR2—RI Set, NEFA HR2—R2 Set and NEFA Standard test kits available from Fujifilm Wako Chemicals Europe under the respective references 434-91795, 436-91995 and 270-77000.

Each of these kits is based on the production of a colored compound. Thus, the respective concentrations of glycerol and non-esterified fatty acids are determined by means of optical absorbance measurements, and the comparison of these measurements with known absorbance ranges for glycerol and non-esterified fatty acid concentrations. The values are normalized by DNA quantification by means of the Quant-iT® PicoGreen® (Invitrogen®) kit available from ThermoFisher Scientific under the reference P7589.

The statistical analysis is performed under the same conditions as for the cytotoxicity study (I).

Adipocytes coming from humans are used for the lipolysis study.

The three adipose tissue donors are adult females respectively 34, 40 and 41 years old undergoing a cosmetic or reconstructive surgery intervention. The donors had a respective body mass index (BMI) of 31.1, 23.3 and 22.9.

The adipocytes are prepared from subcutaneous adipose tissue digested by collagenase followed by a low centrifugation.

The adipocytes are incubated in a Krebs-Ringer bicarbonate (KRB) buffer solution, sometimes containing basolateral medium of caco-2 cells exposed with, either predetermined concentrations of extract(s) (at a rate of one 1:8 dilution each), or with reference products. The incubation time is 2 h at a temperature of 37° C. The cell culture medium is subsequently harvested and frozen at a temperature of −20° C. for use in the glycerol and NEFA tests. The adipocytes are also harvested for the DNA quantification.

Application of Studies I to V on Plant Extracts and Combinations of Plant Extracts

The Applicant conducted investigations on over 100 different plant species. The investigations revealed a great deal of information from which it will be cited:
information about the industrial availability of the plants;
information about the raw material accessibility of the plants;
information about the status and regulations in force for the plants; and
information about the marketing of the plants.

The Applicant retained 25 plant species of which there are Vigna angularis commonly known as adzuki bean or red bean from Japan (seed), Jasminum officinalis commonly known as white jasmine (flower), or also Aframomum melegueta commonly known as Guinea pepper (crushed seed).

In a first test series the Applicant evaluated the cytotoxicity and the effects on adipocyte differentiation and lipid accumulation (adipogenesis), beige adipocyte phenotype (thermogenesis), energy metabolism and lipolysis in relation with the bioavailable extracts coming from these 25 plant species.

The Applicant retained 8 plant species. This concerns the species Nelumbo nucifera commonly known as lotus, Mangifera indica commonly known as Mango, Acacia catechu commonly known as cutch tree, Commiphora mukul commonly known as guggul, Daucus carota commonly known as carrot, Cinnamomum verum commonly known as Ceylon cinnamon tree, Rosa canina commonly known as the dog rose, and Helichrysum italicum commonly known as curry plant.

More particularly, this concerns edible extracts of these species. This particularly concerns the crushed lotus flower, the crushed mango skin, the cutch tree bark, the crushed guggul exudate, the carrot seed, the crushed Ceylon cinnamon tree leaf, the fruit (rose hip) of the dog rose and the aerial part of the curry plant.

The edible extracts of the invention, or of at least the compounds or molecules constituting these extracts, may be designated as active ingredients. In extension, when the edible extracts undergo one or more transformations in order to be conditioned or formulated for the agrifood market, these transformed extracts may be designated as active ingredients.

The fruit of Rosa canina is commonly known as rose hip or cynarrhodium. In botany, this fruit is generally designated as “false fruit”. It comes from the transformation of the floral receptacle of the rose bush and of the dog rose. More generally it comes from plants of the Rosa genus, of the family of Rosaceae.

According to one embodiment, the edible extract of Rosa canina comes from the fruit or from the “false fruit” of this plant.

According to one embodiment of the invention, the edible extract of Helichrysum italicum comes from the aerial part of this plant, that is to say here the stem(s)/flower(s)/leaf(leaves) assembly.

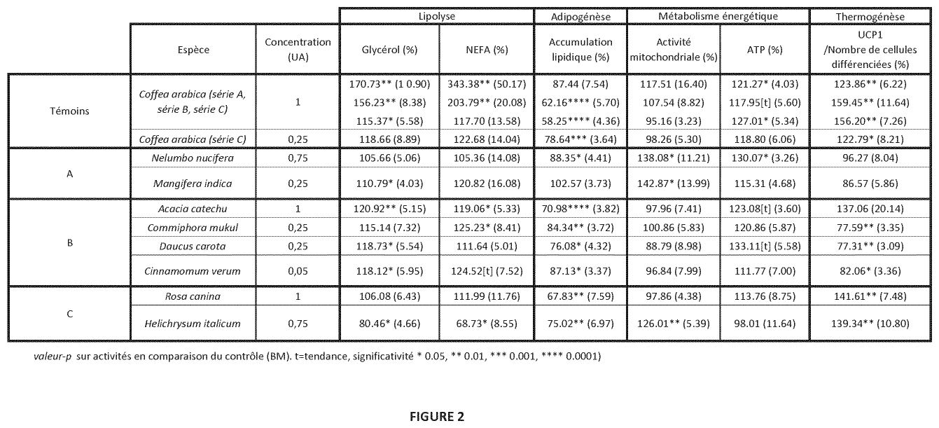

The table in FIG. 2 shows the results relating to the cytotoxicity, preadipocyte differentiation and lipid accumulation (adipogenesis), adipose tissue phenotypic orientation (thermogenesis), energy metabolism and lipolysis of the bioavailable extracts of these eight plant species.

The first test series were broken down into three subseries, noted A, B and C in FIG. 2. Each series is accompanied with control extracts coming from the seed of the species Coffea arabica commonly known as green coffee, cf. FIG. 2 respectively Coffea arabica series A, series B and series C.

More precisions relating to the experimental operations (particularly the chemical and biochemical techniques, or also the identity of the positive and/or negative controls) for obtaining the results are detailed below in relation with the combinations of extracts. The experimental conditions are similar.

The concentration is indicated in FIG. 1 in arbitrary unit (AU). The respective concentrations of series A, B and C are indicated in Table I below.

TABLE I
Concentrations of extracts (series A, B and C).

| | AU = 0.25 | AU = 0.5 | AU = 0.75 | AU = 1 |
|---|---|---|---|---|
| Extract | 0.18 | 0.36 | 0.54 | 0.72 |
| concentration | | | | |
| series A (g/l) | | | | |
| Extract | 0.18 | 0.36 | 0.54 | 0.72 |
| concentration | | | | |
| series B (g/l) | | | | |
| Extract | 0.18 | 0.36 | 0.54 | 0.72 |
| concentration | | | | |
| series C (g/l) | | | | |

The results show that the extracts selected are not cytotoxic for the concentrations used in the first test series. A consumption by humans or animals is possible. The morphological analysis of cells and the trypan blue mortality estimation also show results compatible with a consumption by humans or animals.

The lotus extract has a low lipid accumulation and a high activity on the energy metabolism.

The mango extract has a high lipolytic activity and an activity on the energy metabolism (cf. mitochondrial activity).

The cutch tree extract has a high lipolytic activity and a low lipid accumulation.

The guggul extract has a high lipolytic activity and a low lipid accumulation.

The carrot extract has a lipolytic activity and a low lipid accumulation.

The Ceylon cinnamon tree extract has a high lipolytic activity and a low lipid accumulation.

The dog rose extract has a low lipid accumulation and a high UCPI expression (marker of thermogenesis).

The curry plant extract has a low lipid accumulation, an activity on the energy metabolism (cf. mitochondrial activity), and a high UCPI expression (marker of thermogenesis).

In a second test series, the Applicant evaluated the cytotoxicity, lipid accumulation, cellular differentiation, energy metabolism and lipolysis of bioavailable extracts coming from the combination of a plurality of these extracts.

In particular, the Applicant evaluated the cytotoxicity of six different combinations, each combination comprising two extracts selected from the eight extracts retained in the first test series.

The combinations 1 to 6 are defined in Table II below.

TABLE II
Combination of plant extracts

| Combination no. | Species |
|---|---|
| 1 | Acacia catechu |
| | Commiphora mukul |
| 2 | Acacia catechu |
| | Helichrysum italicum |
| 3 | Acacia catechu |
| | Helichrysum italicum |
| 4 | Daucus carota |
| | Rosa canina |
| 5 | Daucus carota |
| | Rosa canina |
| 6 | Daucus carota |
| | Cinnamomum verum |

Cytotoxicity Study Results (I) of Combinations of Extracts

The basolateral medium (BM) comprising the bioavailable extracts of the Caco-2 cell system is harvested. The preadipocytes are exposed to this BM medium for the cytotoxicity test. Furthermore, a control with respective BM media without extracts is performed, as well as a base control with the FBS 1% base medium. A treatment of the preadipocytes with 0.2% triton is performed for a maximum cytotoxicity control (100%; lysis control). For the viability, a positive control with FBS 10% medium is further performed.

Table III shows the cytotoxicity results.

TABLE III
Cytotoxicity

| | Cytotoxicity | |
|---|---|---|
| | % Base control (SEM) | |
| Base control | 100.00 | (11.76) |
| Lysis control | 1,252.05** | (218.83) |
| Control 1-6 | 214.68 | (100.70) |
| Control 2-4-5 | 160.19 | (75.10) |
| Control 3 | 104.08 | (57.49) |

| | Cytotoxicity | |
|---|---|---|
| | % Respective control (SEM) | |
| Combination 1 | 39.81 | (24.64) |
| Combination 6 | 40.99 | (17.80) |
| Combination 2 | 69.59 | (22.67) |
| Combination 4 | 87.12 | (33.18) |
| Combination 5 | 77.83 | (29.31) |
| Combination 3 | 216.99 | (81.74) |

The results in Table III are expressed in percentage in relation to the control for the release of LDH. The statistical analyses are performed by placing the BM environments comprising the extracts in relation with the respective BM environments without extracts.

The results show that the combinations are not cytotoxic. Combination no. 3 shows a high percentage in relation to the other combinations. However, the cells exposed to this combination no. 3 do not show significant changes in their morphology. This combination is not cytotoxic.

Table IV shows the viability results.

TABLE IV
Viability

| | Mitochondrial Activity | |
|---|---|---|
| | % Base control (SEM) | |
| Base control | 100.00 | (2.88) |
| Positive control | 129.60** | (2.76) |
| Lysis control | 23.78** | (1.14) |
| Control 1-6 | 76.47** | (3.29) |
| Control 2-4-5 | 77.54** | (2.32) |
| Control 3 | 81.64 | (2.23) |

| | Mitochondrial Activity | |
|---|---|---|
| | % Respective control (SEM) | |
| Combination 1 | 133.88** | (2.19) |
| Combination 6 | 104.13 | (1.50) |
| Combination 2 | 129.59** | (6.02) |
| Combination 4 | 92.28 | (2.71) |
| Combination 5 | 95.77 | (2.79) |
| Combination 3 | 105.53 | (6.41) |

The results in Table IV are expressed in percentage in relation to the control for the mitochondrial activity.

In particular, combinations no. 1 and no. 2 considerably increase the mitochondrial activity.

Each of the six extract combinations no. 1 to no. 6 show results compatible with an ingestion by humans or animals.

Adipose Tissue Preadipocyte Differentiation and Lipid Accumulation Study (II) Results in Cells Exposed to Combinations of Bioavailable Extracts

The basolateral medium (BM) comprising the bioavailable extracts of the Caco-2 cell system is harvested for the test.

Preadipocytes not treated by differentiation mixture (NDIF), as well as preadipocytes treated with a PPARy antagonist (GW9662, available from Sigma-Aldrich under the reference M6191-GW9662 inhibits the differentiation) at a concentration of 0.1 μM, are used for negative controls.

The results are expressed in percentages in relation to the results obtained for respective BM media without extracts. The statistical analyses are performed by placing the BM environments comprising the extracts in relation with the respective BM environments without extracts.

Fluorescence microphotographs are performed to quantify the lipid accumulation (Bodipy®). Visible light microphotographs are performed to analyze the morphological state of the cells.

Table V shows the results relating to the lipid accumulation.

TABLE V
Lipid accumulation

| | Lipid accumulation | |
|---|---|---|
| | % Differentiated (SEM) | |
| Non-differentiated | 5.45* | (2.19) |
| Differentiated | 100.00 | (18.06) |
| GW9662 | 30.59* | (8.25) |
| Control 1-6 | 189.63* | (22.78) |
| Control 2-4-5 | 309.96* | (13.25) |
| Control 3 | 239.17* | (20.24) |

| | Lipid accumulation | |
|---|---|---|
| | % Respective control (SEM) | |
| Combination 1 | 73.21t | (7.64) |
| Combination 6 | 97.90 | (7.06) |
| Combination 2 | 45.52** | (3.82) |
| Combination 4 | 57.05** | (6.17) |
| Combination 5 | 49.57** | (6.11) |
| Combination 3 | 73.65* | (4.57) |

Extract combinations no. 2 to no. 5 show a remarkable tendency to the inhibition of the lipid accumulation.

Adipose Tissue Phenotypic Orientation (Thermogenesis) Study (III) Results in Cells Exposed to Combinations of Bioavailable Extracts.

The basolateral medium (BM) comprising the bioavailable extracts of the Caco-2 cell system is harvested for the test.

The results are expressed in percentages in relation to the results obtained for respective BM media without extracts. The statistical analyses are performed by placing the BM environments comprising the extracts in relation with the respective BM environments without extracts.

Fluorescence microphotographs are performed to quantify the UCP1 expression. Visible light microphotographs are performed to analyze the morphological state of the cells.

The negative controls are performed in the same manner as for the preadipocyte differentiation and lipid accumulation study (II) above (NDIF absence and GW9662 presence).

Table VI shows the results of the normalized UCPI protein expression in relation to the total number of cells.

TABLE VI
UCP1 expression (total cell normalization)

| | UCP1 | |
|---|---|---|
| | % Differentiated (SEM) | |
| Non-differentiated | 77.52 | (9.13) |
| Differentiated | 100.00 | (11.06) |
| GW9662 | 83.38 | (11.95) |
| Control 1-6 | 131.29t | (10.38) |
| Control 2-4-5 | 153.47t | (24.85) |
| Control 3 | 162.70* | (8.68) |

| | UCP1 | |
|---|---|---|
| | % Respective control (SEM) | |
| Combination 1 | 100.50 | (10.27) |
| Combination 6 | 157.76* | (19.51) |
| Combination 2 | 146.69* | (13.67) |
| Combination 4 | 168.81* | (14.52) |
| Combination 5 | 135.54* | (12.72) |
| Combination 3 | 110.38 | (7.76) |

Extract combinations no. 2, no. 4, no. 5 and no. 6 show a remarkable UCP1 expression in the preadipocytes.

Table VII shows the results of the normalized UCPI protein expression in relation to the total number of differentiated cells.

For information purposes, this mode of expression may be wise in presence of compounds that may induce an inhibition of the adipocyte differentiation, because UCPI may mostly only be expressed in the mature adipocytes. This makes it possible to differentiate the effect on the UCPI expression, from that on the adipocyte differentiation.

TABLE VII
UCP1 expression (differentiated cell normalization)

| | UCP1 | |
|---|---|---|
| | % Differentiated (SEM) | |
| Differentiated | 100.00 | (16.19) |
| Control 1-6 | 94.96 | (5.90) |
| Control 2-4-5 | 77.46 | (10.16) |
| Control 3 | 103.03 | (8.24) |

| | UCP1 | |
|---|---|---|
| | % Respective control (SEM) | |
| Combination 1 | 127.22 | (18.64) |
| Combination 6 | 158.55 | (22.67) |
| Combination 2 | 253.42** | (30.17) |
| Combination 4 | 262.13** | (33.54) |
| Combination 5 | 248.51** | (39.69) |
| Combination 3 | 127.12 | (16.87) |

Extract combinations no. 2, no. 4 and no. 5 show a remarkable UCP1 expression in the differentiated preadipocytes.

Adipose Tissue Energy Metabolism Study (IV) Results in Cells Exposed to Combinations of Bioavailable Extracts

The basolateral medium (BM) comprising the bioavailable extracts of the Caco-2 cell system is harvested for the test.

A first negative control is performed in the same manner as for the preadipocyte differentiation and lipid accumulation study (II) above: NDIF absence. A second negative control of ATP production is performed in presence of the mitochondrial toxin rotenone (available from Sigma-Aldrich under the reference R8875) at a concentration of 400 nM (for a period of 2 hours on the differentiated cells).

The results are expressed in percentages in relation to the results obtained for respective BM media without extracts. The statistical analyses are performed by placing the BM environments comprising the extracts in relation with the respective BM environments without extracts.

Table VIII shows the ATP production results.

TABLE VIII
ATP production

| | ATP production | |
|---|---|---|
| | % Differentiated (SEM) | |
| Non-differentiated | 85.15 | (3.85) |
| Differentiated | 100.00 | (6.44) |
| Rotenone | 27.95** | (2.33) |
| Control 1-6 | 181.44** | (7.97) |
| Control 2-4-5 | 201.09** | (9.90) |
| Control 3 | 203.74** | (13.21) |

| | ATP production | |
|---|---|---|
| | % Respective control (SEM) | |
| Combination 1 | 109.29 | (6.40) |
| Combination 6 | 96.83 | (3.42) |
| Combination 2 | 100.20 | (3.35) |
| Combination 4 | 92.11* | (2.60) |
| Combination 5 | 102.96 | (3.51) |
| Combination 3 | 94.20 | (3.04) |

The results show that the ATP production is not significantly impaired for cells exposed to the extract combinations no. 1 to no. 6.

Lipolysis Study (V) Results in Cells Exposed to Combinations of Bioavailable Extracts

The basolateral medium (BM) comprising the bioavailable extracts of the Caco-2 cell system is harvested for the test.

Forskolin (1 μM, available from Sigma-Aldrich under the reference F6886) and isoproterenol (0.1 μM, available from Sigma-Aldrich under the reference 16504) are used as lipolysis activators. Forskolin is a compound coming from the plant Coleus forskohlii causing lipolysis by activating adenylyl cyclase. Isoproterenol is an agonist pharmacological compound of the β-adrenergic receptor. Isoproterenol causes lipolysis by activating the β-adrenergic receptors which leads to the activation of adenylyl cyclase. Adenylyl cyclase produces cyclic adenosine monophosphate which is an intracellular messenger triggering a cascade of biochemical reactions that leads to the activation of lipolysis.

The results are expressed in percentages in relation to the results obtained for respective BM media without extracts. The statistical analyses are performed by placing the BM environments comprising the extracts in relation with the respective BM environments without extracts.

Table IX shows the results relating to the glycerol measurements. Table X shows the results relating to the NEFA measurements.

TABLE IX
Glycerol measurements

| | Glycerol | |
|---|---|---|
| | % Differentiated (SEM) | |
| Base control | 100.00 | (2.02) |
| Forskolin | 151.62** | (4.82) |
| Isoproterenol | 213.97** | (17.87) |
| Control 1-6 | 70.81** | (4.96) |
| Control 2-4-5 | 77.23** | (3.56) |
| Control 3 | 72.27** | (6.78) |

| | Glycerol | |
|---|---|---|
| | % Respective control (SEM) | |
| Combination 1 | 98.26 | (4.79) |
| Combination 6 | 115.72 | (8.29) |
| Combination 2 | 74.38** | (4.45) |
| Combination 4 | 118.24* | (7.71) |
| Combination 5 | 92.66 | (2.75) |
| Combination 3 | 86.87 | (6.64) |

TABLE X
NEFA measurements

| | NEFA | |
|---|---|---|
| | % Differentiated (SEM) | |
| Base control | 100.00 | (2.11) |
| Forskolin | 179.70** | (12.42) |
| Isoproterenol | 241.48** | (33.73) |
| Control 1-6 | 70.78 | (12.44) |
| Control 2-4-5 | 66.87* | (8.32) |
| Control 3 | 62.29* | (9.34) |

| | NEFA | |
|---|---|---|
| | % Respective control (SEM) | |
| Combination 1 | 91.20 | (8.40) |
| Combination 6 | 93.89 | (11.07) |
| Combination 2 | 35.38** | (7.51) |
| Combination 4 | 88.38 | (7.49) |
| Combination 5 | 74.74** | (5.56) |
| Combination 3 | 51.13* | (14.53) |

The results particularly show that combination no. 4 increases the release of glycerol.

As a result of all of the results above, combinations no. 1 to no. 6 are combinations of nature to effectively treat overweight or obese subjects.

In a third and fourth series, the Applicant was interested in the combination of respectively three and four bioavailable extracts selected from the bioavailable extracts of Nelumbo nucifera, Mangifera indica, Acacia catechu, Commiphora mukul, Daucus carota, Cinnamomum verum, Rosa canina, and Helichrysum italicum.

The results of the triple or quadruple combination are promising as regards at least one from the cytotoxicity, preadipocyte differentiation and lipid accumulation (adipogenesis), adipose tissue phenotypical orientation (thermogenesis), energy metabolism or lipolysis.

Formulations of the composition of the invention including three or four edible plant extracts selected from Nelumbo nucifera, Mangifera indica, Acacia catechu, Commiphora mukul, Daucus carota, Cinnamomum verum, Rosa canina, and Helichrysum italicum are therefore provided.

Generally, any one of the combinations of 2 of the 8 extracts seems to have a such a good synergy effect that the results are better for at least one of the evaluations II to V above in relation to isolated extracts.

Furthermore, any one of the combinations of 3 or 4 of the 8 extracts seems to have a such a good synergy effect that the results are better for at least one of the evaluations II to V above in relation to isolated extracts.

In a particularly preferred mode, the composition of the invention includes any one of the combinations of 3 or 4 of the edible plant extracts selected from Acacia catechu, Daucus carota, Rosa canina and Helichrysum italicum.

A particular advantage of the invention resides in the fact that the extracts or combinations of extracts identified by the Applicant have an effect on thermogenesis. The composition may therefore be administered per os at any time. Taking meals close together is not determining for obtaining the effects of the composition of the invention.

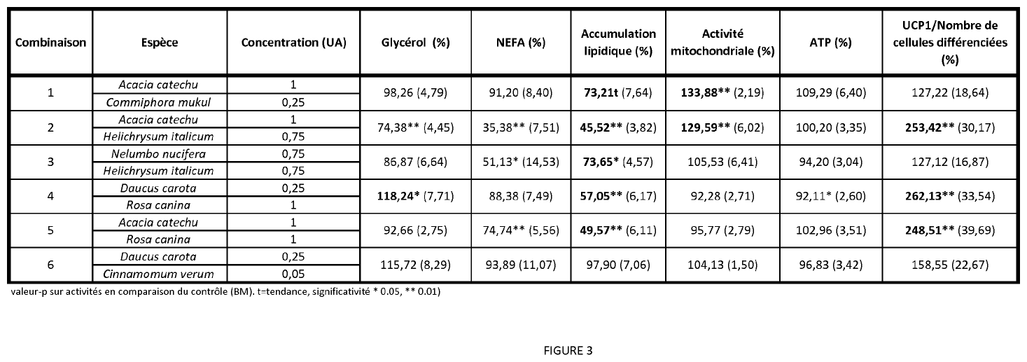

The table in FIG. 3 shows a collection of the results relating to the cytotoxicity, preadipocyte differentiation and lipid accumulation (adipogenesis), adipose tissue phenotypic orientation (thermogenesis), energy metabolism and lipolysis of the combinations of bioavailable extracts according to the invention.

In one example of embodiment, the food supplement composition may be in the form of a 1 g tablet. In this example the contents in ingredients are the following:
plant edible extract(s): 750 to 800 mg;
microcrystalline cellulose: 114 to 164 mg;
sodium croscarmellose: 40 mg;
silicon dioxide: 10 mg;
magnesium stearate: 6 mg;
hydroxypropyl methylcellulose coating: 30 mg.

In a preferable mode, this tablet includes a combination of two edible plant extracts. In a particularly preferred mode, this combination consists of 1% to 99%, preferably 5% to 95%, still more preferably 10% to 90% of a first extract and of 1% to 99%, preferably 5% to 95%, still more preferably 10% to 90% of a second extract, the first and second extracts being selected from Nelumbo nucifera, Mangifera indica, Acacia catechu, Commiphora mukul, Daucus carota, Cinnamomum verum, Rosa canina, and Helichrysum italicum.

In another embodiment, the tablet includes a combination of two edible plant extracts at a rate of 50% each.

In a particularly preferred mode, this combination of two plant extracts consists of approximately 15% to 25% of Daucus carota and of approximately 75% to 85% of Rosa canina. More generally, the combination consists of approximately 15% to 85% of Daucus carota and of approximately 15% to 85% of Rosa canina. For example, the combination may consist of approximately 50% of Daucus carota and of approximately 50% of Rosa canina.

In another preferred mode, the combination of two plant extracts consists of approximately 15% to 85% of Acacia catechu and of approximately 15% to 85% of Commiphora mukul. For example, the combination may consist of approximately 50% of Acacia catechu and of approximately 50% of Commiphora mukul.

In another particularly preferred mode, the combination of two plant extracts consists of approximately 15% to 85% of Acacia catechu and of approximately 15% to 85% of Helichrysum italicum. For example, the combination may consist of approximately 50% of Acacia catechu and of approximately 50% of Helichrysum italicum.

In another particularly preferred mode, the combination of two plant extracts consists of approximately 15% to 85% of Nelumho nucifera and of approximately 15% to 85% of Helichrysum italicum. For example, the combination may consist of approximately 50% of Nelumho nucifera and of approximately 50% of Helichrysum italicum.

In another particularly preferred mode, the combination of two plant extracts consists of approximately 15% to 85% of Acacia catechu and of approximately 15% to 85% of Rosa canina. For example, the combination may consist of approximately 50% of Acacia catechu and of approximately 50% of Rosa canina.

In another particularly preferred mode, the combination of two plant extracts consists of approximately 15% to 85% of Daucus carota and of approximately 15% to 85% of Cinnamomum verum. For example, the combination may consist of approximately 50% of Daucus carota and of approximately 50% of Cinnamomum verum.

The combinations of extracts:
of Daucus carota and of Rosa canina;
of Acacia catechu and of Helichrysum italicum; and
of Acacia catechu and Rosa canina,

have a high activity in thermogenesis stimulation.

In one particular mode, this tablet consists of:
250 mg of edible extract of Daucus carota (seed);
550 mg of edible extract of Rosa canina (rose hip);
microcrystalline cellulose: 114 mg;
sodium croscarmellose: 40 mg;
silicon dioxide: 10 mg;
magnesium stearate: 6 mg;
hydroxypropyl methylcellulose coating: 30 mg

In another embodiment, this tablet consists of:
400 mg of edible extract of Daucus carota (seed);
400 mg of edible extract of Rosa canina (rose hip);
microcrystalline cellulose: 114 mg;
sodium croscarmellose: 40 mg;
silicon dioxide: 10 mg;
magnesium stearate: 6 mg;
hydroxypropyl methylcellulose coating: 30 mg

In one embodiment, the composition of the invention is in the form of a gelatin capsule of size “0”. This type of gelatin capsules has a filler capacity of approximately 300 to 450 mg. In this embodiment, each gelatin capsule comprises:
magnesium stearate when this concerns powder/dry products: 5 to 10 mg; or
silica (silicon dioxide) when this concerns fat powders: 5 to 10 mg; and
approximately 300 to 400 mg of active ingredient.

In another embodiment, the composition of the invention is in the form of a tablet comprising:
magnesium stearate when this concerns powder/dry products: 5 to 10 mg; or
silica (silicon dioxide) when this concerns fat powders: 5 to 10 mg; and
dicalcium phosphate (DCP): 10 to 80 mg;
acacia gum or microcrystalline cellulose: 200 to 300 mg; and
approximately 200 to 300 mg of active ingredient.

In another embodiment the composition of the invention is in the form of a gelatin capsule comprising:
300 mg of active ingredient;
95 mg of a mixture of hydroxymethylcellulose and chlorophyllin;
89.5 mg of long-grain white rice flour;
4.5 mg of magnesium stearate; and
6 mg of rice husk concentrate.

More generally, tablets comprising the composition of the invention may comprise 1% to 99% of active ingredient (plant extract(s)) and the rest in excipients and additives, for example 0 to 10% of magnesium stearate and 0 to 80% of maltodextrin.

More generally, gelatin capsules comprising the composition of the invention may comprise 1% to 99% of active ingredient and the rest in excipients and additives, for example 0 to 10% of silica and 0 to 80% of maltodextrin.

More generally, orodispersible sticks (sachets of dustless powder and ready to be ingested directly or diluted beforehand in a glass of water) or gel sticks (sachet of viscous liquid) comprising the composition of the invention may comprise 0.1% to 70% of active ingredient and the rest in excipients and additives and optionally of aromatic agents and granulation agents.

The composition of the invention may be in the form of orofilms (films melting on the tongue) or of powder to be diluted whereof the quantity of active ingredient may reach up to for example 100%. The composition of the invention may further be in the form of chewing gum whereof the quantity of active ingredient may reach up to for example 50% or also in the form of shot drink (beverage in small portion, generally approximately 10 to 25 cl) whereof the quantity of active ingredient may reach up to for example 30%.

More generally, the composition may be in any usual form of the food industry or of the pharmaceutical industry.

Within the scope of an exploitation of the invention in the pharmaceutical field, the composition may be defined in particular as a composition comprising an edible extract of at least one of the plants selected from the group consisting of Nelumbo nucifera, Mangifera indica, Acacia catechu, Commiphora mukul, Daucus carota, Cinnamomum verum, Rosa canina, and Helichrysum italicum for a use as a medicinal product.

Within the scope of an exploitation of the invention in the medical field, the composition may be defined in particular as a composition comprising an edible extract of at least one of the plants selected from the group consisting of Nelumbo nucifera, Mangifera indica, Acacia catechu, Commiphora mukul, Daucus carota, Cinnamomum verum, Rosa canina, and Helichrysum italicum for a use in a medical method.

The composition and/or the product of the invention defined in the claims makes it possible in particular to activate thermogenesis in a human or animal subject. More generally, the composition and/or the product of the invention defined in the claims may be qualified as modulator of adipose tissue or as fat burner.

In other terms, the composition and the products derived therefrom are modulators of adipose tissue or fat burners, in particular by targeted activation of thermogenesis in a human or animal subject.

1. Oral composition comprising an edible extract of at least two of the plants selected from the group consisting of Acacia catechu, Daucus carota, Rosa canina and Helichrysum italicum.

2. The oral composition according to claim 1, comprising an edible extract of at least three of said plants.

3. The oral composition according to claim 1 comprising an edible extract of all four of said plants.

4. The oral composition according claim 1, comprising a combination of extracts of plants selected from:
a combination of Acacia catechu and Helichrysum italicum;
a combination of Daucus carota and Rosa canina;
a combination of Acacia catechu and Rosa canina.

5. The oral composition according to claim 1, wherein said edible extract comes respectively:
from the bark of Acacia catechu;
from the seed of Daucus carota;
from the fruit of Rosa canina;
from the aerial part of Helichrysum italicum.

6. The oral composition according to claim 1, for use in the treatment and/or the prevention of a disease selected from overweight, obesity and a metabolic disease in a human or an animal.

7. The oral composition according to claim 1, wherein said edible extract is in powder or granular form in which a particle size diameter d90 of particles of the powder or granular form is less than or equal to 180 μm.

8. The oral composition according to claim 1, comprising 0.001% to 99% by weight of the edible extract or extracts.

9. The oral composition according to claim 1, in a form selected from the group consisting of: a tablet, a cachet, a capsule, a granule, a gelatin capsule, a sugar-coated tablet, a chewing gum, a paste, a beverage, a syrup and a powder.

10. Foodstuff, or food supplement, or dietary supplement, or meal replacement product, or beverage, or beverage supplement, or pharmaceutical product, comprising the oral composition according to claim 1.

11. The pharmaceutical product according to claim 10, further comprising one or more supports and/or excipients.

12. The oral composition according to claim 1 for activating thermogenesis in a human or animal subject.

13. Non-therapeutic method for controlling the body weight of a subject, said method comprising the administration of an effective quantity of an oral composition according to claim 1.
